(19) United States
(12) Patent Application Publication
(10) Pub. No.: US 2010/0241026 A1
(43) Pub. Date: Sep. 23, 2010

(54) Portable suction device

(76) Inventor: Alexander Boukas, Sefauket, NY (US)

Correspondence Address: Thomas A. O'Rourke;Bodner & O'Rourke, 425 Broadhollow Road, Melville, NY, 11747, US

(21) Appl. No.: 11/475,855

(22) Filed: Jun. 27, 2006

(51) Int. Cl. A61B 10/00; A61B 5/00; A61M 1/00; C12Q 1/04

(52) U.S. Cl. 600/562; 600/576; 604/319; 435/34

(57) ABSTRACT

An improved suction apparatus, which may be used by health care providers, emergency medical teams, or a corpsmen, to remove debris, fluid and other foreign matter from the airways and/or wounds of injured persons. The suction apparatus which includes: a venturi tube having a forward end, a rear end and an inlet, a valve located on the top rear end portion of the venturi tube, a body for housing at least one can of propellant or compressed gas, an airtight cap, and a trigger mechanism. The improved suction apparatus that forces the propellant passed the inlet of the venturi tube, wherein the inlet is located toward the distal end of the cap.

### FIELD OF INVENTION

The present invention relates to a portable suction device, in particular a portable suction device, which may be used for, cleaning and removing debris associated with wounds, rapidly clearing fluid from airways for easier intubations and collecting and isolating powders, liquids, and gases for rapid screening tests for specific agents.

### BACKGROUND OF THE INVENTION

This application is a continuation in part of U.S. patent application Ser. No. 10/632,134, the contents of which are herein incorporated by its reference.

There is a present need for a device that can collect and/or remove unwanted matter. For example a doctor or emergency service person may need to remove fluid or unwanted matter from a wound area. Or, another example may where a poison control technician may have to collect suspicious matter. One will briefly discuss the need of the present invention with regards to the former example and then discuss the need of the present invention with the latter example.

Fluid buildup in the lungs, pulmonary edema, can be a life threatening condition. Pulmonary edema is generally suspected due to findings in the medical history and physical examination: end-inspiratory crackles during auscultation (listening to the breathing through a stethoscope) can be due to pulmonary edema. The diagnosis is confirmed on X-ray of the lungs, which shows increased vascular filling and fluid in the alveolar walls. In addition, low oxygen saturation and disturbed arterial blood gas readings may strengthen the diagnosis and provide grounds for various forms of treatment. There can be many causes pulmonary edema. Pulmonary edema is either due to direct damage to the tissue or as a result of inadequate functioning of the heart or circulatory system. Generally pulmonary edema is categorized into two classifications, cardiogenic causes and non-cardiogenic causes, better known as ARDS (acute respiratory distress syndrome). The cardiogenic causes are generally caused by: heart failure, tachy- or bradyarrhythmias, severe heart attack, hypertensive crisis, excess body fluids, e.g. from kidney failure, and pericardial effusion with tamponade. The non-cardiogenic causes, or hereinafter, ARDS generally include: inhalation of toxic gases, multiple blood transfusions, severe infection, pulmonary contusion, i.e. high-energy trauma, multitrauma, i.e. severe car accident, neurogenic, i.e. cerebrovascular accident (CVA), aspiration, i.e. gastric fluid or in case of drowning, certain types of medication, upper airway obstruction, reexpansion, i.e. postpneumonectomy or large volume thoracentesis, reperfusion injury, i.e. postpulmonary thromboendartectomy or lung transplantation, and lack of proper altitude acclimatization. Therapies include, when circulatory causes have led to pulmonary edema, treatment with nitrates (nitroglycerien), positive pressure oxygen, and loop diuretics, such as furosemide or bumetanide, is the mainstay of therapy. Secondly, one can start with noninvasive ventilation. Other useful treatments include glyceryl trinitrate, CPAP and oxygen. There are no causal therapies for direct tissue damage; removal of the causes (e.g. treating an infection) is the most important measure.

Most forms pulmonary edema, those associated with cardiogenic causes, is readily treatable with a variety of medications. However, those forms of pulmonary edema caused by complication of ARDS, which symptoms include difficulty breathing, coughing up blood, excessive sweating, anxiety and pale skin, if left untreated, can lead to death. It is this type of pulmonary edema that has taken the life's of so many. As mentioned above ARDS, may be caused by an accidental trauma, as in a car accident, or ARDS may be the result of an intentionally inflicted wound, as in combat. Whether, in combat or any emergency situation, when one is afflicted with ARDS, time is of the essence, it is imperative that the response team or corpsman be able to administer oxygen to the afflicted. However, in more severe cases, intubations may be accompanied by mechanical respirations and a ventilator. In order to facilitate intubations, physicians and corpsman alike, need a portable suction device to remove objects, such as shattered teeth and other foreign matter, and fluids such as plasma, pleural fluid, saliva, blood, and secretions, that may become lodged in the airway or airspace of the injured. In addition, physicians and/or corpsman may need to excise a bullet fragment, and maintain a clear surgical field. In this example however the bulky oversized equipment, such as compressors, associated with most hospital setting would not be a practical tool to implement. If one was equipped with a portable suction device, as in the present invention, the need for such bulky equipment would not be necessary.

In addition to removing foreign matter from wounds and/or air passageways the present invention can also be used to collect, contain and test different matter. The need for such a device is ever increasing especially with the constant threat of bioterrorist attacks. The matter that terrorist may use to implement their diabolical schemes may be in liquid, gas or solid form. There are a variety of different pathogens that bioterrorists may use Bacterium, Viruses, Protozoa and Fungi are but a few. One example of a bacterial pathogen is anthrax, a popular toxin among bioterrorists, it generally comes in a solid form, i.e. a powder. In the United States in the year 2001, over the course of several weeks beginning on Sep. 18, 2001 (after the Sep. 11, 2001 attacks) several letters containing anthrax bacteria were mailed to several news media offices and two U.S. Senators, killing five people. With the United States increasing their efforts on the current war on terror it is only a matter of time before the next terror attack will occur.

Bioterrorists may also use proteins to facilitate their terror campaign. For example, the Ricin protein is a toxin from the castor bean. Ricin can be extracted from castor beans and is known to have an average lethal dose in humans of 0.2 milligrams ( 1/5,00^{th} of a gram), though some sources give higher figures, Ricin is considered twice as deadly as cobra venom. Ricin is poisonous if inhaled, injected, or ingested, acting as a toxin by the inhibition of protein synthesis, making it ideal for a bioterrorist attack.

The present invention relies on principles based, in most part, on Bernoulli's continuity equation and the Venturi meter. According to the continuity equation, the speed of fluid flow can vary along of the paths of the fluid. The pressure can also vary; it depends on the speed of flow. From Bernoulli's continuity equation p₁+½ρυ₁²=p₂+½ρυ₂², and, υ₂=(A₁/A₂)υ₁. Where ρ is the density of the liquid, υ₁ and υ₂ are the velocities at points 1 and 2 respectively, p₁ and p₂ are the respective pressures, and A₁ and A₂ are the respective areas. The Venturi meter, which is used to measure flow speed in a pipe, has a narrow part called a throat, A₂, and a wide part, A₁, and two inlets to measure the pressure at points 1 and 2. When fluid flows through the venturi meter, because A₁ is greater than A₂, υ₂ is greater than υ₁ and the pressure p₂ in the throat is less than p₁. A net force to the right accelerates the fluid as it enters the throat, and a net force to the left slows it as it leaves, this is known as the Venturi effect.

This principle has been further developed throughout the years. For instance rather than use a venturi meter to measure fluid flow, one may implement a venturi tube to create a vacuum, as in the present invention. A venturi tube is a tube that has a restricted portion with an inlet located thereto; the restricted portion acts as the throat in the venturi meter. The venturi tube utilizes similar principles of fluid flow and pressure to create a vacuum, with one additional concept; when the pressure at any given point is less than atmospheric pressure, it is called a vacuum pressure, a vacuum. Thus, if one were to allow the pressure, p₂, at point 2, in the venturi meter to fall below atmospheric pressure than a vacuum would be formed. Hence, one may form a suction device via a venturi tube with an inlet located at the confined portion.

To sum up, the Venturi effect is a special case of the Bernoulli effect, in the case of fluid flow or air flow through a tube or pipe with a constriction in it. The fluid must speed up in the restriction, reducing its pressure and producing a partial vacuum via the Bernoulli effect. A venturi tube to form a vacuum has been implemented in the past, as seen in U.S. Pat. No. 6,094,778, issued to Boukas, and U.S. Pat. No. 6,845,542, assigned to the research Foundation of State University of New York, Albany N.Y. In the above prior art the venturi tube is attached to a container, which houses an aerosol propellant. In addition the venturi tube has opening at both ends with an inlet located at the confined portion of the tube. When the propellant is released through the venturi tube a vacuum is formed at the inlet, thus creating a vacuum and allowing for removal of any debris or fluid. The debris or fluid can also be collected, contained and tested via an attachment comprising of a filter, a testing strip and a buffering agent, located inside and at the front end of the venturi tube.

### OBJECTS OF INVENTION

It is an object of the present invention to provide a fluid apparatus for rapidly clearing fluid from an airway or wound site.

It is an object of the present invention to provide a fluid removal apparatus which can be held in a single hand and is human powered.

It is another object of the present invention to provide a fluid removal apparatus which is portable, cost effective and easy to maintain.

It is still another object of the present invention to provide a fluid removal apparatus for removing excess fluids from a surgical field to allow doctors and pre-hospital care personal to operate more effectively.

It is yet another object of the present invention to provide a fluid removal apparatus with increased propellant flow.

It is still another object of the present invention to provide a fluid removal apparatus that may be connected to a separate tank of compressed gas so as to allow for extended use in mass casualty situations.

It is another object of the present invention to provide an attachment for the fluid removal apparatus that may be easily connected.

It is still another object of the present invention to provide an attachment for the fluid removal apparatus that may be operated by doctors, pre-hospital care personal, disease control technicians and those not skilled in the art.

It is yet another object of the present invention to provide an attachment for the fluid removal apparatus that may detect a multitude of different pathogens, toxins and the like.

It is still another object of the present invention to provide an attachment for the fluid removal apparatus that implements a filter that can be used to contain matter in a solid, liquid or a gas state.

It is still yet another object of the present invention to provide an attachment for the fluid removal apparatus that can give a positive or negative test result for the contained matter in a rapid efficient manner.

It is still a further object of the present invention to provide an attachment for the fluid removal apparatus that is disposable after a test is performed.

It is yet another object of the present invention to provide an attachment for the fluid removal apparatus that may be reused after a test is performed.

### SUMMARY OF INVENTION

The present invention implements a venturi tube configuration for the suctioning of foreign unwanted matter, from airways or airspaces and wounds from trauma victims. In one embodiment the suction device comprises a generally cylindrical cavity. The cavity includes an inside and outside surfaces for maintaining a propellant or compressed gas. In addition, the outside wall may have a trigger mechanism located on the bottom portion of the suction device, and a tube that may be attached to the outside surface of the suction device. The suction device further comprises a release valve, which is connected to an airtight pocket. The airtight pocket receives and forces the compressed gas or propellant to the export duct, which sends the compressed gas or propellant to the venturi tube, wherein a vacuum is created.

In a similar embodiment the suction device comprises, a trigger that is located on the top portion of the suction device, wherein the trigger performs the same function as the trigger in the previous embodiment. The trigger in this embodiment is either attached to the release valve or rests on the release valve. The suction device further comprises a release valve, which is connected to an airtight pocket. The airtight pocket receives and forces the compressed gas or propellant to the export duct, which sends the compressed gas or propellant to the venturi tube, wherein a vacuum is created.

In all embodiments a multitude of different attachments may be implemented with the present invention, from suctioning tools to containment bags. In addition, any of the aforementioned embodiments may also include a collection, isolation and testing attachment, which may be used for rapid screening of toxins, pathogens and the like. The collection device may include an immunoassay test strip, which is a biochemical test that measures the level of a substance in a biological liquid, typically serum or urine, using the reaction of an antibody or antibodies to its antigen. The addition of this collection attachment allows the user to collect and isolate suspicious foreign matter. It is especially useful in the identification of toxins and/or pathogens used by bioterrorist agents, it achieves this by implementing the immunoassay test strip located in the flexible hose portion of the attachment.

One of the main disadvantages of the prior art is that the duration of propellant flow through the venturi tube is limited. As the propellant in the canister changes from a liquid to a gas when flowing out, it requires and absorbs heat from the remaining liquid and immediate area. As the canister cools, the vapor pressure of the propellant greatly decreases, thus causing some of the propellant to remain in the canister, which reduces the outward flow of propellant and diminishes the venturi effect. The present invention improves on the prior art by implementing different methods of heating the canister of propellant or compressed gas. Either by using an outside heating source, heat sink, or by adding a PCM or other additive, such as water

In all the embodiments the inlet of the venturi tube is located near the rear end portion of the suction device.

### BRIEF DESCRIPTION OF INVENTION

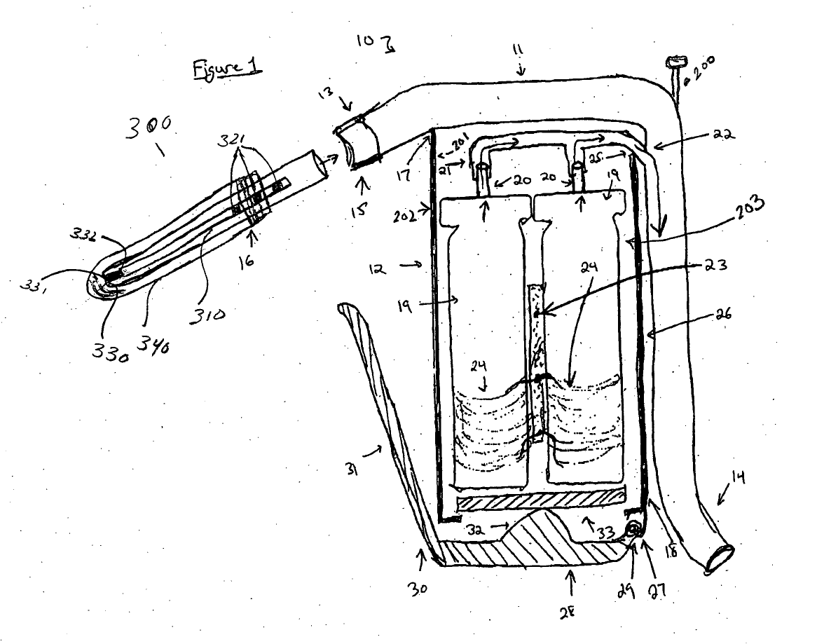

FIG. 1 is a front vertical cross-sectional view of canister embodiment of the present invention.

FIG. 2 is a vertical cross-sectional view of a single-canister embodiment of the present invention.

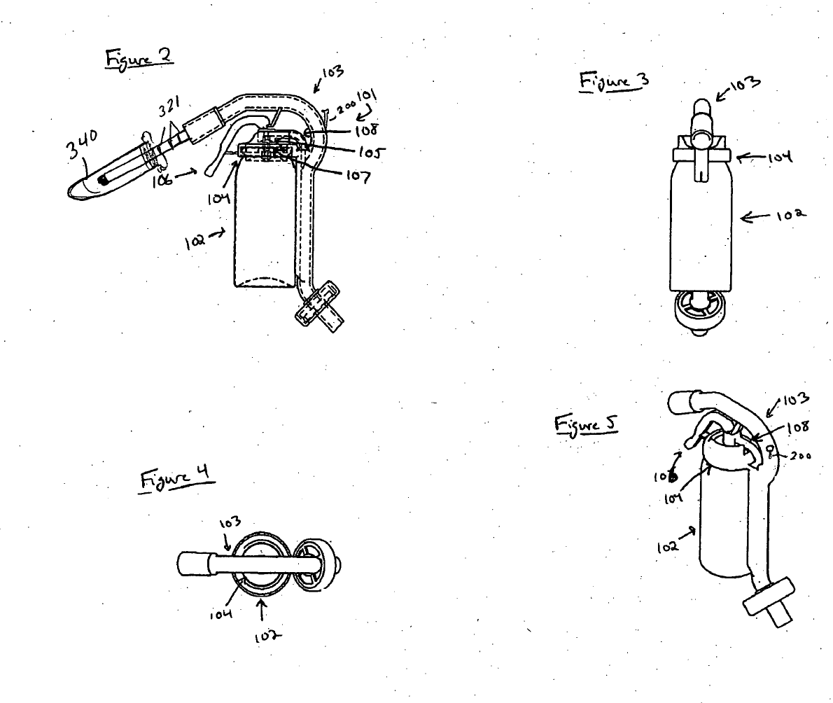

FIG. 3 is a front view of the device shown in FIG. 2.

FIG. 4 is a top view of the device shown in FIG. 2.

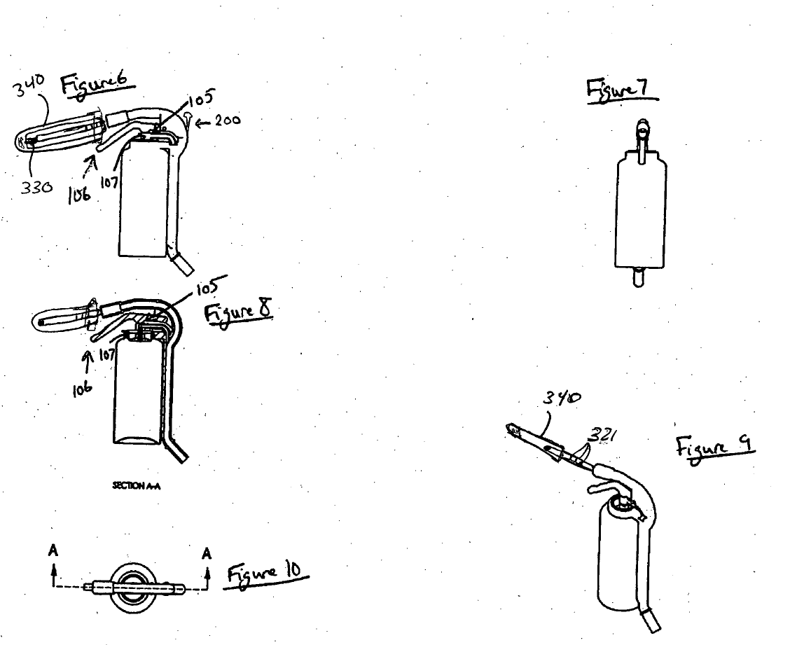

FIG. 5 is a rear perspective view of the device shown in FIG. 2.

FIG. 6 is a side view of a second embodiment of a single-canister version of the present invention.

FIG. 7 is a front view of the embodiment shown in FIG. 6.

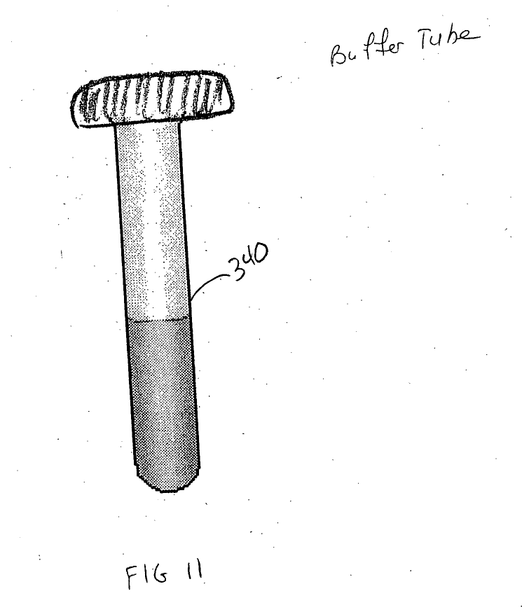

FIG. 8 is a cross-sectional view of the embodiment shown in FIG. 6.

FIG. 9 is a rear perspective view of the embodiment shown in FIG. 6.

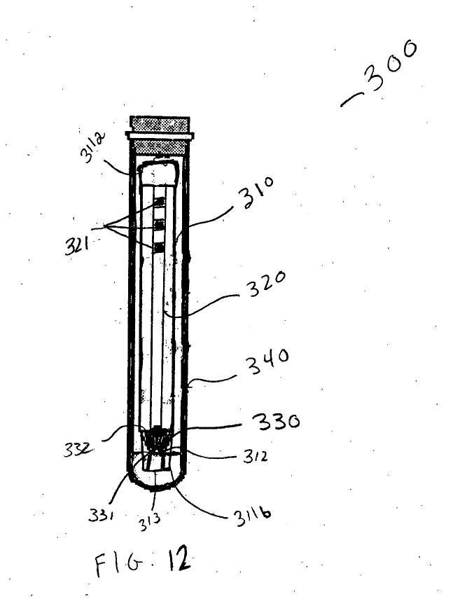

FIG. 10 is a top view of the embodiment shown in FIG. 6.

FIG. 11 is a perspective view of a test tube containing a buffer solution.

FIG. 12 is a perspective view of the immunoassay test strip inside of the test tube with a buffer solution.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

In FIG. 1, the fluid removal apparatus is generally shown at 10. The venturi tube 11 may be of any length (hereinafter tube). Tube 11 may be constructed of any suitable material known in the art capable of handling gases, liquids, or small particles. In the present embodiment the tube 11 was constructed of a clear flexible plastic. Tube 11 may be attached to body 12 by any suitable known attachment method in the art, but not limited to Velcro, adhesive, or adhesive tapes. Tube 11 has a forward end 13 and a rear end 14. In one embodiment tube 11 may have a valve 200 located on the outside rear-end portion 201 of hose 11. Valve 200 may act as a connection device for additional equipment, such as an external compressed gas container. Tube 11 may have an attachment coupling 15 located on forward end 13. Any type of suitable attachment coupling may be used, but not limited to a ring or sleeve that creates a frictional fit between the inner surface of the sleeve or ring and the outer surface of the tube. Conversely one may use an adhesive to attach coupling 15 to tube 11. Coupling 15 may be constructed of any suitable known material in the art but not limited to rubber, glass, metal, metal alloy, or plastic. In the present embodiment coupling 15 was constructed of plastic. There are a variety of different attachments that may be coupled to hose 11. In the present embodiment an elongated hose with a means for removing debris was implemented. Coupling 15 may provide a vacuum seal between hose 11 and attachment device.

In one embodiment, as seen in FIG. 1, tube 11 may be permanently attached to body 12, either on top surface 17 and /or side surface 18. In another embodiment, tube 11 may be only attached to body 12 when vacuum device is being used. This may allow for cleaning and replacement of tube 11.

Body 12, as seen in FIG. 1, is of generally cylindrical shape, however any suitable known shape in the art may be implemented. Body 12 has inside and outside surfaces, 201 and 202 respectively, which form cavity 203. Cavity 203 may be of such dimensions so as to be able to house one or multiple cans of propellant. In the present embodiment cavity 203 was constructed to house two cans of propellant 19. In addition to housing propellant, body 12 may be adapted to receive a variety of different types of propellant, such as compressed gas or aerosol. Furthermore, body 12 may be designed to harness propellant 19 in a multitude of methods, i.e. friction fit, threaded fit, or any other type of fastening means that are associated with aerosol or compressed gas cans. In the present embodiment body 12 was adapted to receive propellant via a frictional fit. Depending on the nature of the intended use, the suction apparatus may be designed as a one shot disposable device or a rechargeable multi use device. In the present embodiment propellant 19 was readably replaceable when fully discharged. In addition, one may attach an outside source of compressed gas to valve 200 in situations where it is not practical to continuously replace individual cans of propellant; e.g. in mass casualty situations, where constant changing of cans of propellant would waste valuable time, one could hook up a large tank of compressed gas such as oxygen to the portable fluid removal apparatus. Each can of propellant 19 have a release valve 20, which is connected to tube 11 by an export duct 21. When activated, the release valves feed compressed gas into export duct 21, which passes into tube 11 at inlet or port 22. The export duct may be made of any suitable tubing material for handling a gaseous material. In addition export duct 21 extends longitudinally toward rear end of suction device 10.

As mentioned above, one of the disadvantages of the prior art is that the venturi effect is diminished during discharge do to heat loss. Based on fundamental thermodynamic principles, the amount of heat added to effect the various phase changes is equal to the change in enthalpy; the change of enthalpy between a liquid and a vapor phase is the latent heat of vaporization. As the propellant in the canister changes from a liquid to a gas, it requires and absorbs heat from the remaining liquid and the immediate area. When the propellant is released canister 22 cools, thus decreasing the internal pressure of the canister. The end result is excess propellant remaining in the canister. In order to compensate for this heat loss, one may implement an additional outside heat source, as seen in FIG. 1. Between the cans of propellant 10 can be a battery 23. The battery 23 may be connected to resistor wire 24 which is wrapped around each can. When activated, the current in the resistor wire heats the cans to increase the pressure of the gas inside the cans and thus increase the suction created. Heat sensitive fuse blocks (not shown) may be employed to prevent overheating.

In another embodiment one may implement a heat sink to heat the canister of propellant. Generally, a heat sink is an environment or object capable of absorbing heat from another object with which it is in thermal contact (either direct contact or radiational contact). In common use, it is a device made of metal brought into contact with the hot surface of an object. In this type of embodiment one could wrap aluminum or copper, or any other good thermal conductor, around the outside surface of the canister and create a reverse heat sink, i.e. one could use the ambient temperature to heat the canister.

In yet another embodiment one may implement a PCM in the can of compressed gas. The PCM should be a material that is capable of latent heat storage. A PCM is a material that will stay relatively the same temperature during phase change. For example, PCM's absorb and retain heat when changing from solid to liquid, but release heat when changing from a liquid to solid. Some PCM's that can be implemented with the can of compressed gas are paraffin waxes, normal paraffin, and Fischer-Tropsch hard waxes. Preferably, the PCMS have a melting point between about −3° Celsius and 100° Celsius. When compressed gas is expelled from the can and the temperature decreases, the PCM will release heat, which may keep the suction effect constant.

In still another embodiment, in order to maintain an enough heat to produce total vaporization of the propellant, one may mix the desired propellant with an additive. For example, in one embodiment, one may use a propellant such as tetrafluoroethane, R134a. However, any suitable known propellant in the art including but not limited to CO₂, as in the preferred embodiment, Argon and the like may be implemented. CO₂ was the preferred propellant, because of it's non-flammability, and higher pressure, which can increase suction. During discharge, as the R134a in the canister changes from a liquid to a gas, it requires and absorbs heat from the remaining liquid and the immediate area, the latent heat of vaporization for R134a is 216.8 kJ/kg. As the canister cools, the vapor pressure of the propellant greatly decreases which reduces the outward flow and diminishes the venturi effect to zero in about 15 seconds (assuming 3.5 ounces of R134a in canister). This results in ˜100 ml of water suctioned up into a collection bag, and about 1 ounce of R134a left in the container. As mentioned previously, one may implement any of the above methods for increasing heat to increase internal pressure. However, one may also use an additive to increase the latent heat of vaporization. In the present embodiment equal parts of pure water and R134a were mixed, which resulted in ˜200 ml of water being suctioned up into the collection bag and an additional ˜1.8 ounces of R134a being vaporized. There are a multitude of propellants and additives that may be implemented to achieve similar results, and as such this embodiment is for illustrative purpose and is should not be limited to the above suggestions.

In one embodiment activation means 26 runs along side surface 18 of body 12. At one end 25, the activation means 26 may be in the vicinity of or attached to export duct 21. At a second end 27, the activation means 26 may be attached to a first end 29 of a trigger 28. At a second end 30 of trigger 28 is a depression means 31. Depression means 31 may be molded as a hand grip, with a finger indentation to allow the user to grip and squeeze easily. The trigger 28 is operated as a lever with a fulcrum 32 resting, or hingedly attached, to the bottom surface 33 of body 12.

FIG. 2 depicts another embodiment of the present invention at 101. In this embodiment, only a single can of compressed gas 102 is preferably utilized. While the tube 103 placement is similar to the previous embodiment, the release of compressed gas differs.

Device 101 may attach to a can of compressed propellant 102 by an airtight cap 104. Cap 104 encapsulates the release valve 105 on the top of can of compressed propellant 102. A trigger mechanism 106 similar, to the trigger mechanism in our previous embodiment, may be implemented. Trigger 106 may attach to release valve 105, as seen in FIGS. 2-5, or trigger 106 may rest on release valve 105 as seen in FIGS. 6-10. In either embodiment trigger 106 is depressed via the user's digit, thus releasing the propellant out of the canister and into airtight pocket 107. Pocket 107 maintains airtight integrity, which forces the propellant out through export duct 108 into venturi tube 103. Export duct 108 is adjacent to pocket 107 and longitudinally extends toward the rear end of suction device 101, as in previous embodiments. Venturi tube 103 is of basic design; a tube comprising of a conduit and an inlet. In simplest form when a fluid moves through an inlet, a low pressure develops at the inlet, if the pressure is sufficiently low relative to the external pressure; a partial vacuum is created, as mentioned previously. In the prior art the venturi device is located on the top forward end of the suction device. In the present invention, the inlet of the venturi tube is located toward the distal end of cap 104, near the rear end of suction device 101.

It is understood that the body, tube, and can of propellant or compressed gas may be of any size, shape, or material. Preferably, the tube will be soft plastic and the body will be hard plastic for easy maintenance and cleaning. Additionally, the body may have a clip for attachment top a belt or strap of a bag. Also, in the present invention the cans of propellant or compressed gas are easily replaceable, by either removing them from the body, or removing the clip.

In any embodiment, a container (not shown) may be attached to rear end 14 of tube 11. Preferably, the container is a bag or pouch that will enable the user to collect any fluid or debris that is suctioned through tube 11. The container may have an open end, which will preferably create an airtight seal around rear end 14 of tube 11. In some manner, the bag may allow gas to escape, while suctioning liquid, debris or polluted air. In one embodiment the container will be equipped with an attachment port, which can mate with a filter. The filter may allow gas to escape, while retaining liquid, debris, or polluted air. In a second embodiment, the container itself may be manufactured from a gas-permeable, liquid retaining compound. In a further embodiment, the container may be equipped with vents that will allow gas to escape. In each embodiment, the container may be equipped with a sealing means, which will allow the user to remove and seal the container. A sealing means, which may be an adhesive strip or cap, can be used if the container houses hazardous material, or a liquid that can be reused after being filtered.

As mentioned previously the present invention may include a variety of different attachments, such as a collection, isolation and test attachment 300. Attachment 300 may further include flexible hose 310, immunoassay test strip 320, filter portion 330 and test tube buffer 340, as seen in FIG. 12.

Drawing one's attention again to FIG.12 is hose 310. Hose 310 may have a front end 311b and a rear end 311a. Rear end 311a may be any suitable shape known in the art. For example rear end 311a can be circular, square or rectangular in shape. The shape of rear end 311a will depend on the shape of the couple used to attach collection device 300 to one of the aforementioned suction devices. In a preferred embodiment one used a generally rounded shape for rear end 311a. Rear end 311a may be connected to any of the aforementioned suction devices via any suitable connection methods known in the art including but not limited to press fitting, screwing and the like. With any connection methods used rear end 311a may be either the male or female end. In a preferred embodiment rear end 311a was press fitted into the suction device, i.e. rear end 311a was the male end, this ensures that no collected matter can escape between the outside coupling surface and the inside surface of rear end 311a. Front end 311b may be generally circular in shape, however any suitable shape known in the art may be used. In the preferred embodiment front end 311b has aperture 313. Aperture 313 may be any suitable shape known in the art, including but not limited to a square, rectangle and the like. Aperture 313 may taper off in a direction toward filter portion 330, also seen in FIG. 12, this ensures that the gathered matter is confined to filter portion 330. Also aperture 313 may have a diameter equal to or less then front end 331 of filter 330. Filter 330 may be any suitable type of filter known in the industry. In the preferred embodiment a highly dense non-reactive material was used. The length and amount of filter 330 will depend on the desired use, i.e. the type of matter being collected, contained and tested. Filter 330 may have a front end 331 and a rear end 332. Front end 331 of filter 330, as mentioned above is adjacent to front end 331a of hose 310, this ensures that the matter being collected and contained does not get exhausted out the rear.

Inside of hose 310 may be immunoassay test strip 320. In one embodiment test strip 320 may be attached to rear end 332 of filter 330 and extend the entire length of hose 310, or only partially therefrom. In the preferred embodiment test strip 320 was adjacent to rear 332, but not attached and it extended the entire length of flexible hose 310. Test strip 320 may have one color indicator, or many color indicators, this will also depend on the matter to be collected. In the preferred embodiment one used a test strip with 3 color bands 321, as may be seen in FIG. 12. Test strip 320 can be designed to detect a multitude of different toxins and/or pathogens, in the preferred embodiment it was desirous to detect the presence of Anthrax and Ricin.

As mentioned previously attachment 300 may include a test tube 340. Test tube 340 may be seen in more detail in FIG. 11. Test tube 340 may any suitable test tube shape known in the art. It shall be pointed out now that in the preferred embodiment test tube 340 will fit snuggly around hose 310. To ensure this snug fit one used a test tube with a tapered inside surface. Test tube 340 may also contain a buffer solution, the type of solution used will depend on the type of chemical reaction desired. For example, there can be one type of buffer solution for detecting Anthrax and a different type of buffer solution to detect Ricin.

In normal operation, when the user desires to collect, isolate and test suspicious foreign matter, the user will connect attachment 300 to one of the aforementioned suction devices. The user will then place the suction device in the vicinity of the suspicious foreign matter. The user will then activate the suction device in a manner described previously. The vacuum created by the suction device will then pull the suspicious foreign matter up through aperture 313 of tube 310. As the foreign matter is being sucked up it will become trapped into anterior filter 330. The user will then remove the buffer test tube cap and place it over tube 310. The buffer solution will then be contact anterior filter 330 where it will dissolve and stabilize the trapped foreign matter. The reactive solution will then be absorbed up through test strip 320. If a toxin and/or pathogen are present it will cause one of the color indicators to develop. If a toxin and/or pathogen are not present then the color indicators will not develop.

As various changes could be made in the above constructions without departing from the scope of the invention, it is intended that all matter contained in the above description shall be interpreted as illustrative and not in a limiting sense. In the view above it will be seen that several objects of the invention are achieved and other advantageous results attained.

We claim:

1. A matter removal and collection apparatus comprising
a venturi tube having a forward end and a rear end;
a body having at least one can of propellant therein and an opening for releasing said propellant, said venturi tube being attached to said opening in said body, said venturi tube being located on rear end of said body; and
a trigger means attached to said body, and said trigger means being adapted to cause said propellant to be released from said body thereby causing a suction effect at said forward end of said venturi tube by regulating the flow of propellant out of said rear end of said tube;
an attachment for collecting, isolating and testing matter, wherein an attachment is connected to said forward end of said venturi tube.

2. A fluid removal apparatus according to claim 1, which said forward end of said tube, is fitted with a coupling for securing an attachment thereto.

3. A fluid removal apparatus according to claim 1, wherein there are at least two cans of propellant.

4. A fluid removal apparatus according to claim 1, wherein said cans are connected by an export duct.

5. A fluid removal apparatus according to claim 1, wherein said propellant is a mixture of equal parts water and tetrafluorethane.

6. A fluid removal apparatus according to claim 1, wherein said propellant is a mixture of equal parts water and CO₂.

7. A fluid removal apparatus according to claim 1, wherein said propellant is a mixture of equal parts water and Argon.

8. A fluid removal apparatus according to claim 1, wherein said body has a battery, said battery is connected to a resistor wire, said resistor wire being wrapped around said can of propellant.

9. A fluid removal apparatus according to any of claim 5, 6, or 7 wherein said body has heat sensitive blocks to prevent overheating.

10. A fluid removal apparatus for patient treatment device comprising:
a venturi tube having a forward and rear end;
a cap attaching said tube to a can of compressed gas; and
a trigger attached to said cap, said trigger adapted to control a suction effect at said forward end of said venturi tube by regulating the flow of propellant from said cans of propellant out of said rear end of said venturi tube;
an attachment for collecting, isolating and testing matter, wherein an attachment is connected to said forward end of said venturi tube.

11. A fluid removal apparatus according to claim 10, wherein said forward end of said tube is fitted with a coupling for an attachment.

12. A fluid removal apparatus according to claim 10, wherein said cap makes an airtight seal with said can of propellant.

13. A fluid removal apparatus according to claim 10, wherein said can of propellant is connected to said tube by an export duct.

14. A fluid removal apparatus according to claim 10, wherein said can of propellant has a release valve.

15. A fluid removal apparatus according to claim 14, wherein said release valve is controlled by said trigger.

16. A hand-held, self contained fluid removal apparatus comprising:
a venturi tube having a forward and rear end;
a cap attaching said venturi tube to a can of compressed gas;
a trigger attached to said cap, said trigger adapted to control a suction effect at said forward end of said venturi tube by regulating the flow of compressed propellant from said cans of propellant out of said rear end of said tube; and,
a container attached to said rear end of said tube;
an attachment for collecting, isolating and testing matter, wherein an attachment is connected to said forward end of said venturi tube.

17. A container for receiving gases, fluids, and solids comprising:
an open end;
an attachment port on a surface of said container; and
a filter removably attached to said port, said filter being adapted to allow gas to escape while inhibiting the release of liquid, polluted gas and solids.

18. A container according to claim 17, wherein said open end is adapted to create an airtight seal.

19. A container according to claim 17, wherein said open end has a sealing means which allows the container to be sealed airtightly.

20. A container according to claim 19, wherein said sealing means is an adhesive strip.

21. A container according to claim 19, wherein said sealing means is a cap adapted to fit airtightly over said open end of said container.

22. A fluid removal apparatus for patient treatment comprising
a venturi tube having a forward end and a rear end;
a body having at least one can of propellant therein and an opening for releasing said propellant, said venturi tube being attached to said opening in said body, said venturi tube being located on rear end of said body; and
a trigger means attached to said body, and said trigger means being adapted to cause said propellant to be released from said body thereby causing a suction effect at said forward end of said venturi tube by regulating the flow of propellant out of said rear end of said tube;
an attachment for collecting, isolating and testing matter, wherein an attachment is connected to said forward end of said venturi tube.

23. A fluid removal apparatus according to claim 22, wherein on the top rear end of said venturi tube is a valve adapted to receive an outside source of compressed gas.

24. A method of removing fluids, gases, or solids from the airway or wound site of a patient comprising:
positioning a tube in said airway or said wound site, said tube being attached to a fluid removal apparatus which comprises a tube having a forward end and a rear end a cap attaching said tube to a can of compressed gas, and a trigger attached to said cap, said trigger adapted to control a suction effect at said forward end of said tube by regulating the flow of compressed gas from said cans of compressed gas out of said rear end of said tube;
applying pressure to said trigger to create a suction effect at said front end of said tube; and,
suctioning liquids, gases, or solids from the airway or wound site;
collecting, isolating and testing matter, wherein an attachment is connected to said forward end of said venturi tube.

25. A method of removing fluids, gases, or solids from the airway or wound site of a patient comprising:
positioning a tube in said airway or said wound site, said tube being attached to a fluid removal apparatus which comprised a tube having a forward end and a rear end a cap attaching said tube to a can of compressed gas, a trigger attached to said cap, said trigger adapted to control a suction effect at said forward end of said tube by regulating the flow of compressed gas from said cans of compressed gas out of said rear end of said tube, cans of compressed gas out of said rear end of said tube, and a container attached to said rear end of said tube;
applying pressure to said trigger to create a suction effect at said front end of said tube; and,
suctioning liquids, gases, or solids from said airway or said wound site.
collecting, isolating and testing matter, wherein an attachment is connected to said forward end of said venturi tube.

26. A fluid removal apparatus for patient treatment comprising
a venturi tube having a forward end and a rear end;
a body having at least one can of propellant therein and an opening for releasing said propellant, said venturi tube being attached to said opening in said body, said venturi tube being located on rear end of said body;
a trigger means attached to said body, and said trigger means being adapted to cause said propellant to be released from said body thereby causing a suction effect at said forward end of said venturi tube by regulating the flow of propellant out of said rear end of said tube;
an attachment for collecting, isolating and testing matter, wherein said attachment is connected to said forward end of said venturi tube.

27. The matter removal and collection apparatus according to claim 26 wherein said attachment further comprises:
a flexible hose having a front end and a rear end, said rear end being inserted into said front end of said front end of said venturi tube, said front end of said hose having an aperture, said aperture having a front end and a rear end, said rear end of said aperture having a filter adjacent to it, said flexible tube also having a test strip located inside of said tube, said filter having a front end and a rear end said test strip being located between said rear end of said filter and said rear end of said tube; and
a test tube having a buffer solution, said buffer solution being able to dissolve and stabilize toxins and pathogens.

28. The matter removal and collection apparatus according to claim 27 wherein said test strip has at least one color indicatator.

29. The matter removal and collection apparatus according to claim 28 wherein said buffer solution is dissolves and stabilizes the toxin Anthrax.

30. The matter removal and collection apparatus according to claim 28 wherein said buffer solution is dissolves and stabilizes the toxin Ricin.
